(19) United States
(12) Patent Application Publication
(10) Pub. No.: US 2025/0022558 A1
(43) Pub. Date: Jan. 16, 2025

(54) SYSTEMS AND METHODS FOR GENERATING STRUCTURED RECORDS BASED ON MEDICAL IMAGES OF AN ENDOSCOPY

(71) Applicant: Iterative Scopes, Inc., Cambridge, MA (US)

(72) Inventors: Jonathan Ng, Cambridge, MA (US); Daniel Wang, Cambridge, MA (US)

(21) Appl. No.: 18/770,286

(22) Filed: Jul. 11, 2024

Related U.S. Application Data
(60) Provisional application No. 63/512,952, filed on Jul. 11, 2023.

(51) Int. Cl. G16H 10/60; A61B 1/00; G06T 7/00

(52) U.S. Cl. CPC G16H 10/60; A61B 1/000096; G06T 7/0014; G06T 2207/10068; G06T 2207/20081; G06T 2207/30092; G06T 2207/30096

(57) ABSTRACT

This specification describes systems and methods for generating structured data records, such as electronic health records (EHR) or electronic medical records (EMR), based on medical data generated during an endoscopy procedure. More specifically, the data processing system and methods described in this document are configured to generate structured medical data automatically from images, videos, associated metadata, and other data generated during an endoscopy procedure for a patient.

### CLAIM OF PRIORITY

This application claims priority under 35 U.S.C. § 119(e) to U.S. Patent Application Ser. No. 63/512,952, filed on Jul. 11, 2023, the entire contents of which are hereby incorporated by reference.

### TECHNICAL FIELD

This disclosure generally relates to systems and methods for scoring and quantifying disease, potential malignancies, such as inflammation, and predicting disease progression and treatment outcomes.

### BACKGROUND

Endoscopy refers to a nonsurgical procedure used to visualize a patient's intestinal tract. An endoscope is a flexible tube with a light and camera that is guided through a patient's digestive tract allowing images (e.g., still images and/or video) of the digestive tract to be taken. There are two broad categories of endoscopy based on site of insertion. During an upper endoscopy, the endoscope is passed through the oral cavity and allows the doctor to visualize the esophagus, stomach, and upper part of the small intestine. During a lower endoscopy, the endoscope is inserted through the rectum to visualize the entire colon, Endoscopic procedures allow physicians to evaluate several medical conditions, such as colorectal cancer and irritable bowel disease and view other abnormal features within the colon such as appearances of ulcers, bleeding in the digestive tract, and detection of potential malignancies (e.g., polyps). Endoscopic procedures are commonly utilized by doctors to measure Inflammatory Bowel Disease (IBD) outcomes and activity and to assess colorectal cancer prevention. The FDA also views endoscopic endpoints (in addition to patient reported symptoms) as a key measure of drug efficacy and performance for IBD-related clinical trials.

Images and/or videos generated during an endoscopy provide record of the anatomical appearance of the intestinal tract and state of disease of the patient. The process of generating a record of the endoscopy procedure can be time consuming for the physician and often results in records that are not uniformly filled out. Generally, a physician reviews the images and/or video taken and populates a structured medical record, such as an electronic medical record (EMR) with details of the procedure. The physician can generate notes that annotate the images and/or video data or supplement context for those data. The physician also documents relevant clinical judgements and impressions based on the data.

### SUMMARY

This specification describes systems and methods for generating structured data records, such as electronic health records (EHR) or electronic medical records (EMR), based on medical data generated by an endoscopy procedure. More specifically, the data processing system and methods described in this document are configured to generate structured medical data automatically from images, videos, associated metadata, and other data generated during an endoscopy procedure for a patient.

A data processing system is configured to obtain video or image data from an endoscopy. This includes medical image data, including video data, as well as metadata associated with the images and/or video data. The data processing system is configured to perform one or more image processing operations to extract relevant data from the video data and label the video data.

In some implementations, the data processing system has standardized requirements with regards to information provided to a third party record. These requirements can include a particular format, a range of values, a type of data, and so forth. These requirements will be determined prior to interaction with a third party record requiring the data. For example, specifications made by a healthcare institution or physician may result in a third party record requiring that the data processing system provide particular medical data related to the endoscopy, such as a number of polyps found, a degree or severity of irritation discovered (e.g., on a given scale), or any other relevant information related to the outcome of an endoscopy. These specifications are determined prior to the creation of the data processing system.

In some implementations, the requirements and specifications for processing the data are determined prior to the creation of the data processing system. The data processing system creates a standardized level of detail that can be accessed in an application through API calls or other methods. One or more different machine learning models are configured to output the respective different details, and the level of detail is standardized throughout the process. In this example, if a medical service provider (e.g., a physician) require amending the level of detail, the record that is automatically generated can be amended to add or remove data that are automatically output by the data processing system.

In some implementations, the data processing system outputs data according to requirements and specifications that are dynamic. These requirements or specifications are able to be updated on the fly (e.g., through an API). In this example, a medical service provided (e.g., a physician) can adjust a level of detail for output records to increase or decrease the level of detail as desired.

In some implementations, the data processing system determines what data is required by a given third party record. The data processing system is flexible in that it determines how to satisfy the requirements of populating the record.

The data processing system analyzes the labeled video and/or image data to generate data that satisfies the requirements of the record (e.g., populates all fields of the record as required for the record). For example, the data processing system may perform an image processing analysis to extract from the video data representing the number of polyps found during the endoscopy. The data processing system may process the video or image data to determine a severity of bleeding or degree of inflammation of the intestinal tract as found from the endoscopy. The data processing system is configured to determine locations of polyps or ulcers in the patient.

The data processing system is configured to generate additional details about the ulcers or polyps in the patient, such as number, size, shape, and description of the polyps or ulcers. The data processing system is configured to recognize tools in the images and/or videos and document a time of appearance in that data. The data processing system is synchronized with metadata associated with the image and video data to tag the extracted features with timestamps and patient particulars.

The data processing system is configured to populate fields that are not related to the endoscopy data itself but are descriptive of the endoscopy. For example, the data processing system can specify timestamps for important procedure points (e.g., start and stop time of endoscopic procedure, time of cecum detection etc.), the identity of the physician/patient, anatomical location of a specified image and/or frame in a video (e.g., anus, rectum, sigmoid colon, descending colon, splenic flexure, transverse colon, hepatic flexure, ascending colon, cecum, appendiceal orifice, ileocecal valve, proximal esophagus, distal esophagus, z-line, diaphragm indentation etc.), a score used to determine presence of and severity of Inflammatory Bowel Disease (e.g., MES score for Ulcerative Colitis, SES-CD for Crohn's disease etc.), specify presence of areas affected, details and severity of diseases (e.g., size, location and quality of diverticulum; size, location, complications and tools used to traverse strictures; size and location of internal hemorrhoids etc.) indicate type of procedure performed, indicate a start/stop location in the endoscopy and other metadata such as what equipment was used for the procedure, indicate or what medical facilities were used (e.g., an operating room number, a hospital name, name of surgeon performing procedure, interventions performed during the procedure (e.g., biopsy, polypectomy, colectomy etc.).

The data processing system is configured to ensure that each field of the structured data record is populated with a value. In some implementations, if a value is unavailable for the population of a field, the data processing system generates an alert or cue that identifies the empty field to a user. Once the record is complete, the data processing system is configured to send the completed record (either as a whole record or in its constituents) to a storage system configured for storing and accessing the structured record. In some implementations, this storage system is in the cloud.

The systems and methods described in this specification provide one or more of the following advantages.

First, the data processing system is configured to generate more robust data records than would otherwise be generated manually by a physician. The image and/or video data and corresponding metadata provide a much more detailed analysis of an endoscopy than what would be generated by a physician working from memory or reviewing the video data. In some implementations, the data processing system provides a second-to-second record of the procedure based on video and/or image data collected. Generally, a physician has a limited time to generate the record of a procedure and may not be able to perform a detailed review of each frame of an endoscope video. Because of this, details of the endoscopy can be lost. The data processing system is configured to retain this data and generate a summary of relevant portions of the data for creating a more robust record of the procedure overall.

Second, the data processing system allows for improved precision and accuracy of documentation. This is contrasted to manual documentation by a physician working from memory or reviewing the video data, reducing lapses in human judgment and human error. Technical processing of image and/or video data will also be able to provide a much more detailed analysis of an endoscopy. In conjunction with the anatomical landmark identifier built into our data processing system, specific features and findings in the colon will be able to be tagged to exact anatomical landmarks, allowing monitoring of disease progression on repeated endoscopy.

Third, the data processing system is configured to reduce time spent by physicians on documentation tasks, thereby allowing for increased time spent on dedicated patient care. Time on clinical documentation can be reduced, lessening their administrative workload and burden of physicians. This frees up time in the clinic and operation room that can be channeled to patient care and treatment, enabling better patient engagement and outcomes. A physician spends up to 35% of his or her time documenting patient data, and this product could allow up to an estimated saving of 20% of their time spent in documentation.

Fourth, the data processing system ensures standardization of medical documentation and reduces intra- and inter-operator variability in documentation of endoscopic procedures by physicians, through the use of standardized data reporting formats and templates. This allows for easier follow-up, progress tracking, and information retrieval, improving the quality of care provided to patients.

The standardized medical documentation includes records with a structure that satisfies data quality rules and that enables computing systems to automatically retrieve details about the patient from the record based on the structure. For example, the data processing system can ensure that each of the fields of the record are populated with data in an expected format to prevent errors for processing the records for one or more applications. For example, the data processing system can ensure that each data record is associated with a valid key value corresponding to a particular patient or consistent with a schema specified for storing the structured data record. For example, each video for a particular patient can be associated with a consistent key value so that all of the data records for that patient can be retrieved from the system. The system can ensure that the key value does not conflict with one or more other key values associated with the patient which may occur when patient records are ingested from one or more target EMR/HER data sources. Additionally, each of the specific features of the structured data record, such as the features extracted from video data, can be associated with the key value in a consistent way so that each of these data items are accessible for analysis or automated analysis of the patient data.

Each of the following embodiments are configured to enable one or more of the foregoing advantages.

In a general aspect, a method for automatically generating a structured medical record from endoscopy data includes obtaining image data including endoscopic images of a gastrointestinal tract (GI) of a patient; determining one or more features to extract from the image data, the features each representing a physical parameter of the GI tract; extracting the one or more features from the image data; processing the features to generate transformed features corresponding to fields of a structured medical record; and storing, in a data store, one or more data entries including the transformed features, wherein the data store is configured to receive structured queries for the data entries in the data store and provide the data entries including the transformed features in response to receiving the structured queries.

In some implementations, extracting the one or more features from the image data comprises: applying a machine learning model to the image data, the machine learning model being trained with images of GI tracts; identifying, from the applying, a malignancy in the GI tract; determining one or more physical features of the malignancy; and outputting the one or more physical features as feature data.

In some implementations, the one or more physical features comprise a color of the malignancy, an outer shape of the malignancy, an area of the malignancy, a location of the malignancy, or an orientation of the malignancy.

In some implementations, the process includes comparing the one or more physical features to corresponding one or more features extracted from other image data; and generating comparative features data.

In some implementations, the structured medical record comprises a set of instances of records each associated with a common identifier. The process can include comparing data of a first field of a first instance of the instances of the records with a second field of a second instance of the instances of the records for generating comparative feature data.

In some implementations, the structured queries comprise a natural language query. The process includes processing the natural language query using a language model trained based on a library of terms associated with the fields of the structured medical record.

These and other aspects, features, and implementations can be expressed as methods, apparatus, systems, components, program products, means or steps for performing a function, and in other ways. These and other aspects, features, and implementations will become apparent from the following descriptions, including the claims.

### BRIEF DESCRIPTION OF THE DRAWINGS

FIG. 1 shows a data processing system for automatically generating structured data records from endoscopy data associated with an endoscopic procedure.

FIG. 2A shows data flow for processing image data and generating EMR data.

FIG. 2B shows an example of a structured data record for EMR data.

FIG. 3 shows an example query from an EMR system and response provided by the data processing system.

FIG. 4 show an example flow diagram for automatically generating structured data records from endoscopy data associated with an endoscopic procedure.

### DETAILED DESCRIPTION

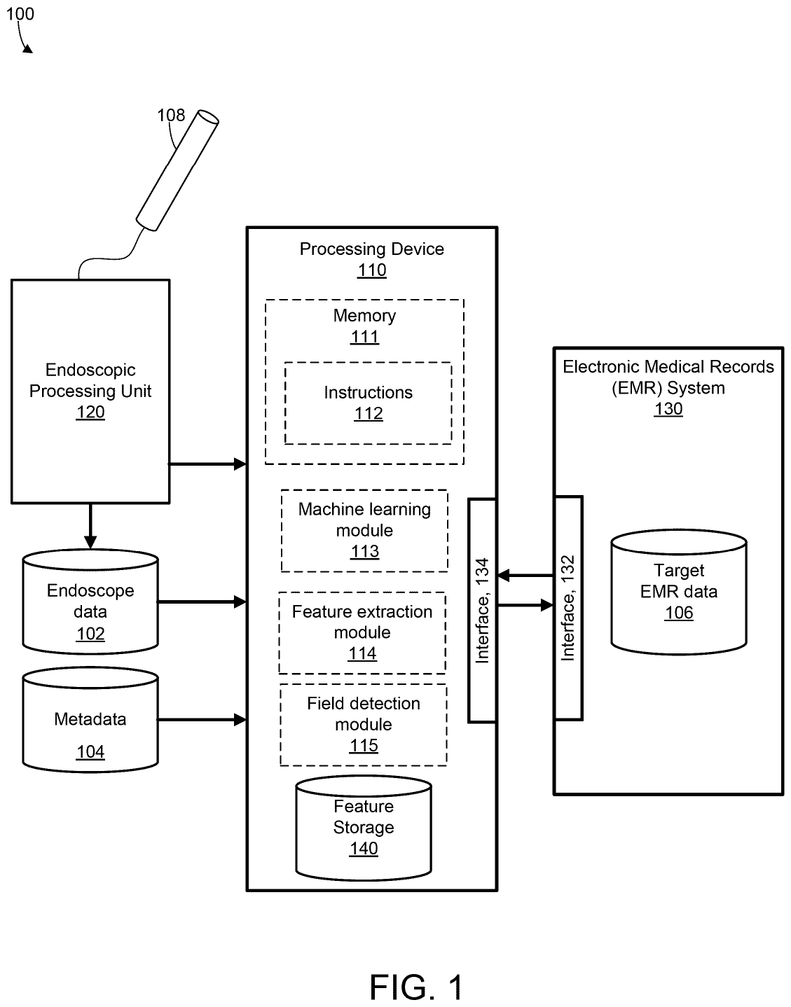

FIG. 1 shows an example of a data processing system 100 configured to execute one or more processes for automatically generating structured data records, such as electronic medical records, from endoscopy data associated with an endoscopic procedure. The data processing system includes a processing device 110, a memory 111 (or a computer readable hardware storage device) configured to host instructions 112, a machine learning module 113 configured to execute feature extraction or identification, image processing module 114 to process, identify and extract features and a field detection module 115. In some implementations, the instructions 112 can be hosted directly in the processing device 110 during instances when they are accessed frequently. Generally, endoscopy data includes data generated from or associated with an endoscopic procedure and can include video data 102 and associated metadata 104. The data processing system 100 can include an endoscopic processing unit 120 configured to connect to an endoscope 108 for capturing image and/or video data generated by an imaging device of the endoscope 108 during the endoscopy.

Endoscopy data generally includes all data generated from the imaging device of the endoscope 108 during an endoscopy and subsequent data (such as feature data) generated from the images or video data. The endoscopy data represents the endoscopy and can be analyzed or processed (e.g., by the processing device 110) for generation of the EMR data 106. The endoscopy data 102 are associated with metadata 104 that describe the endoscopy data 102. For example, the metadata 104 include data/time information, descriptions of the surgical tools being used (such as names or types), a location of the procedure, a name of the physician performing the procedure, and so forth. For example, the data processing system 100 may be able to determine a make and model of the endoscope based on data obtained through the connection to the endoscopic processing unit 120 to the endoscope 108. The metadata 104 can include start time and stop times for a given procedure, and from these data, the processing device 110 determines a duration of the procedure. The metadata 104 may be useful for generating logs or populating portions of the EMR, as subsequently described.

In some implementations, no metadata 104 are provided. Rather, the data processing system 100 is configured to extract features from the video data or image data and generate values for populating metadata 104 fields. For example, the data processing system 100 is configured to detect, from the video, a tool type, a location of the procedure in the GI tract, and so forth. The data processing system 100 is configured to obtain video data 102 from the endoscopic processing unit 120. The endoscopic processing unit 120 includes an imaging device that is configured to capture image data or video data 102. In some implementations, the imaging device is an endoscope 108. The endoscope 108 is an illuminated optical, thin, and tubular instrument (e.g., borescope) used to examine internal organs like the throat or esophagus. The endoscope can be shaped and configured to target specific organs, such as the bladder, kidney, bronchus, colon, and/or pelvis. In some implementations, the endoscope is flexible and includes a camera on one end. The camera can capture image data in the form of still images and/or video. The image or video data 102 can take the form of several data formats, such as RAW, JPEG, PNG, etc. In some implementations, the imaging device includes a digital camera that uses a charge-coupled device (CCD) and/or complementary metal oxide semiconductor (CMOS) to convert photons to electrons for digital processing.

The EMR data 106 includes records associated with individual patients. The EMR data 104 can include self-reported data of the patient. The EMR data 106 can include data obtained from physicians or other medical service providers from interacting with the patient in addition to the data generated automatically by the data processing system 100. For example, the EMR data 106 can include a medical history for the patient, such as medical operations the patient has experienced, illnesses the patient has experienced, and physiological data associated with the patient.

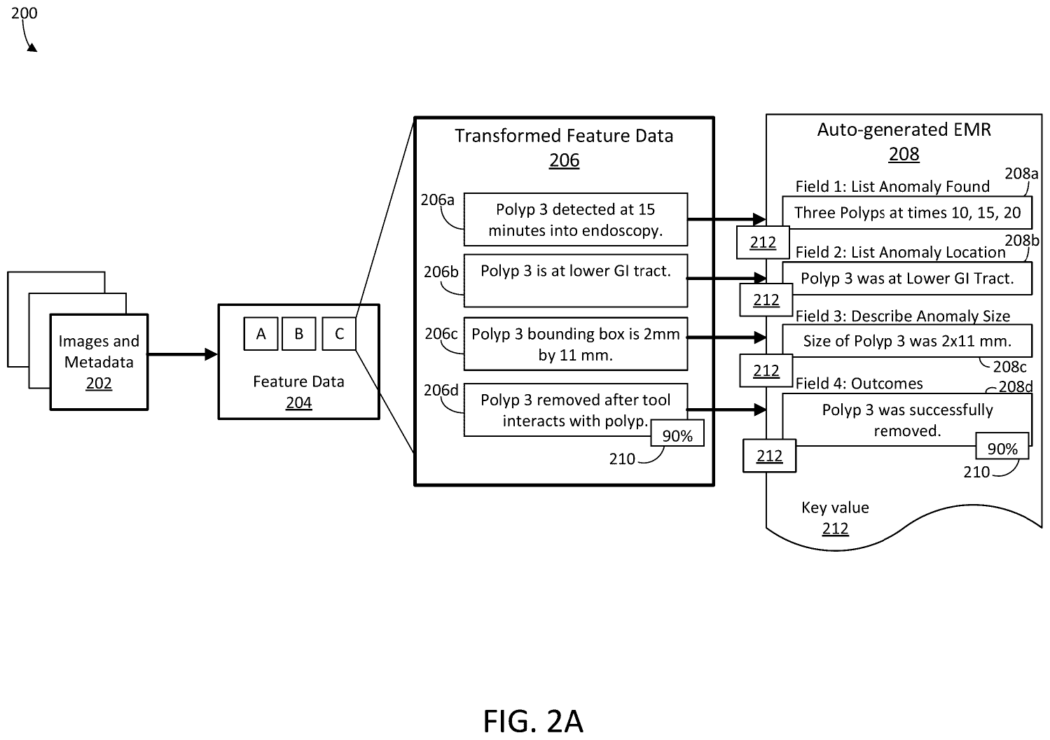

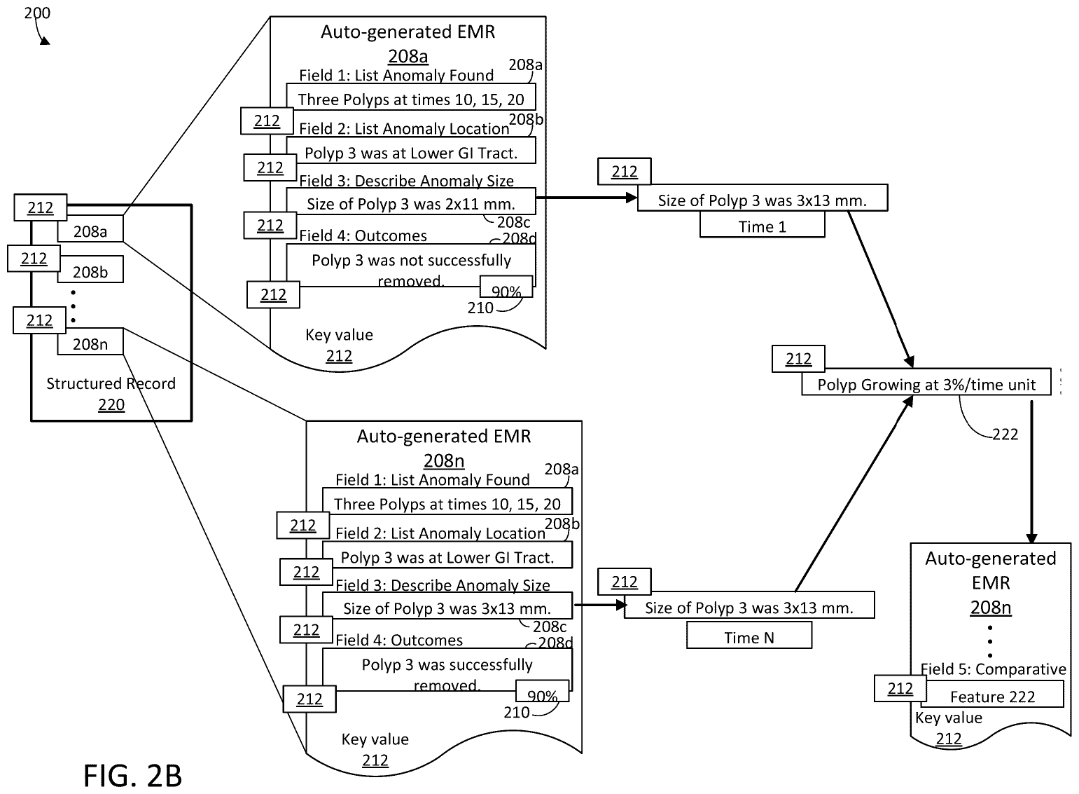

The EMR data 106 generally include a structured record with fields and values in the fields. The EMR data 106 include fields that describe medical aspects of the endoscopy. Different examples of EMR data 106 may include different fields and thus require different combinations of values to be generated by the processing device 110 for populating those fields. For example, a first EMR may include a field for duration of the endoscopy, while a second EMR (e.g., of a different system) may include two fields indicating a start time and a stop time of the endoscopy, but include no field specifying the duration of the procedure. The processing device 110 is configured to determine which fields are included in a given target EMR 106 and populate those fields with the appropriate values automatically. In some implementations, the field may not specify a value acquired directly from the endoscopy data 102 or associated metadata (e.g., start and stop times). Instead, the field may require some additional processing by the processing device 110 to obtain the necessary value, such as calculating a procedure duration from the start and stop times and populating a “duration” field of the target EMR 106 with the duration of the procedure. As such, the processing device 110 is configured to determine what values are specified by fields of the target EMR data 106 and provide the corresponding values. This is described further in relation to FIGS. 2A-2B.

The EMR data 106 include medical records for particular patients. The EMR data 106 can include data that conform to standard forms. The EMR data 106 can include clinical data for a patient that is provided by a medical service provider in response to a patient visit or telehealth interaction. Generally, the EMR data 106 are on a per-patient basis. This provides a rich history for a particular patient.

The EMR data 106 includes fields and corresponding values representing an endoscopy of a patient in addition to other medical information. The EMR 104 can include a field specifying whether medications are being used by the patient. For example, the EMR data 104 can include whether the patient is using diphenoxylate or opiates as anti-diarrheal medication. The EMR data 104 can include one or more fields specifying demographics data such as age, sex, reproductive history, smoking status, and race or ethnicity of the patient. In another example, one or more fields include values representing data obtained from physically examining the patient by a physician. For example, fields can include a patient medical history which may indicate ileocolonic resection, data indicative of one or more of the presence or absence of an anal fissure, a fistula or abscess, and the presence or absence of one or more complications such as uveitis, pyoderma gangernosum, erythema nodosum, and/or arthralgia. One or more fields of the EMR data can include values representing physicians' global assessment of the patient (e.g., indicating the presence or absence of a condition). The EMR data 106 can include values from pathology laboratory results, such as representing serological profiling results for a time period. The EMR data 106 can include values representing a history of medications prescribed to the patient, including current medications and biologics. The EMR data 106 can include values that represent whether the patient has used biologics. The EMR data 106 can include values that represent disease activity (e.g., whether a disease is active or inactive). The EMR data 106 can include values that represent an IBD type, such as whether the type includes UC or CD. The EMR data 106 can include values that represent a discase duration (e.g., in years). The EMR data 106 can include values that represent a history of surgery for the patient (e.g., whether it has occurred, what surgery has occurred, and when surgery has occurred). The EMR data 106 can include values that represent whether steroid-free remission has occurred. The EMR data 106 can include values that represent fistula drainage (e.g., an extent or occurrence). The EMR data 106 can include values that represent whether the patient has experienced pain or activity restriction (e.g., frequency and severity values associated with either or both). The EMR data 106 can include values that represent a degree of induration for the patient. The EMR data 106 can include values that represent a presence or size of an abdominal mass in the patient. The EMR data 106 can include values that represent whether sexual activity has been restricted. The EMR data 106 can include values that represent a history of flaring (e.g., during a study associated with the patient). The EMR data 106 can include values that represent a hospitalization history for the patient (e.g., time, duration, frequency, etc.). The EMR data 106 can include values that represent a history of thrombosis for the patient (e.g., frequency, location, and/or severity).

In another example, the EMR data 106 can include results from the short IBD questionnaire (e.g., an SIBDQ). These fields can include values representing a patient diet, such as whether dairy has been consumed. The EMR data 106 can include values representing environmental exposures of the patient, including whether over the counter (OTC) drugs have been consumed by the patient, patient infections (e.g., types, locations, frequencies, etc.), and whether the patient has traveled or undergone major life events that may contribute stress to the patient's life. The EMR data 106 can include values representing relevant family history of disease. The EMR data 106 can include values representing fecal incontinence in the patient in the past.

In these examples, the processing device 110 is configured to extract features from the image data and/or video data 102 and metadata 104 to populate the fields with values corresponding to the previously described values. The feature extraction module 114 extracts the data based on fields detected by the field detection module 115.

The processing device 110 is configured to receive video or image data 102 from a procedure (e.g., from a colonoscopy). The image or video data 102 generally includes a sequence of frames, each representing a portion of the colon (or other such patient data). A subset of the frames or images of the video or image data 102 can represent symptoms of a disease, such as inflammatory bowel disease (IBD) or other disease. For example, the images can represent bleeding, ulcers or sores, narrowing of the intestines, polyps, and so forth. The processing device 110 is configured to identify the frames or images of the data 102 that represent symptoms using the image processing device 114 (also called a feature extraction module).

The feature extraction module 114 is configured to process the image or video data 102 and other data associated with the endoscopy of the endoscopy data 102 to extract data for populating fields of the target EMR 106. In some implementations, the image processing module 114 is a part of a machine learning module 113, wherein the image processing module extracts data from the images or videos, and the machine learning module performs classification of the extracted data. For example, the image processing module 114 may perform thresholding operations or feature extraction based on signals received from the machine learning module (e.g., setting threshold values or identifying features in the images to extract).

In some implementations, the data processing system 100 performs a non-visual based tagging. The non-visual tagging can include identification of audio data and/or metadata 104 to determine values for fields of the structured data records.

The feature extraction module 114 can process the images or frames of the video data 102 on an individual basis and/or in combination with one another to identify the presence of IBD symptoms, and so forth). For example, the image processing module 114 can process images frame by frame to identify a symptom presence in the frame by signature matching a region of the image to a known signature representing a symptom). In some implementations, the image processing module 114 is configured to identify where in the image the symptom is manifested and identify, to other module (such as the machine learning module) which frames or sequence of frames are associated with a symptom.

The feature extraction module 114 generally is configured to draw bounding boxes or otherwise tag or identify images or frames as representing a symptom. However, how the image processing module 114 identifies the symptoms can be changed or updated based on feedback from the machine learning module 113. For example, the image processing module 114 can extract image data based on thresholds set or adjusted by the machine learning module. In some implementations, the machine learning module is configured to update, based on training data, image signature data used for classification of the image or video data.

The feature extraction module 114 can process groups of frames or images of video data 102 together. The feature extraction module 114 can be configured to analyze the image in the context of a previous frame (or series of frames) or a subsequent frame (or series of frames). The feature extraction module 114 is configured to facilitate extraction and/or recognition, from image data, of features that are used for populating fields of the target EMR 106. For example, the feature extraction module 114 can facilitate detection of bleeding, polyp formation, etc. by applying one or more feature extraction processes using image processing, and populate an EMR field requesting a list of symptoms, a number of polyps detected, and so forth. Image processing processes can include object detection, pixel thresholding, application of filters to the images or portions of the images, and so forth.

The endoscope data 102 includes gastro data describing the patient based on the image data received from endoscopy procedures of the patient. The gastro data can include values that represent a location of the endoscopy, such as a lower GI endoscopy. The gastro data can include values that represent a presence of ulcers and/or a number of ulcers. The gastro data can include values that represent a relative vascularity, such as a percentage decrease of vascularity. The gastro data can include values that represent presence of erosions, and a number of the erosions. The gastro data can include values that represent presence or absence of bleeding in the GI tract, and a number of times bleeding was observed (e.g., a number of frames including evidence of bleeding). The gastro data can include values that represent erythema in GI tract. The gastro data can include values that represent a friability (e.g., in GI tract). The gastro data can include values that represent a size of ulcers or erosions. The gastro data can include values that represent a presence of stenosis (e.g., narrowings) of the GI tract. The gastro data can include values that are associated with an upper GI endoscopy (e.g., that specified as located in the upper GI endoscope data). The gastro data can include values that represent a total ulcerated surface (e.g., presence or absence of this surface, and a percentage of the tract including such a surface). The gastro data can include values that represent a surface affected by disease (e.g., as a percentage of the total surface). The gastro data can include values that represent a disease location in GI tract. The gastro data can include values that represent a number of lesions observed (e.g., at the case level). The gastro data can include values that represent a presence of cobblestoning in the tract. The gastro data can include values that represent a presence of deep ulcers. The gastro data can include values that represent a type of Crohn's disease observed (e.g., non-stricturing, non-penetrating, stricturing, penetrating, stricturing and penetrating, or perianal). The gastro data can include values that represent a presence of dysplasia in the patient. The gastro data can include values that represent whether activity at a biopsy site is proximal or distal. The features of the image data 102 representing the gastro data are used to populate one or more fields of the target EMR 106 based on the fields identified by the field detection module.

The gastro data can accessed as follows. In an example, the gastro data can be included in existing EHR. The gastro data can be detected and extracted from endoscopy videos when converting an image/video to structured gastro data described. For example, video data 102 includes images and videos that represent the GI tract. The data processing system is configured to extract features from the image or video data, label these features as one or more elements of the gastro data described previously, and record these in the EMR system in a searchable format. In this way, the data processing system 100 generates the first instance of the gastro data for the data records.

The feature extraction engine 114 is configured to analyze the metadata 104 to extract data describing the endoscopy video data 102 that is used for populating fields of the target EMR 106. For example, the feature extraction engine 114 receives a list of fields from the field detection module 115. Based on the list of fields, the feature extraction engine is configured to analyze the metadata 104 to extract the particular portions of that data which are useful for populating the fields of the target EMR 106. This is described further in relation to FIGS. 2A-2B, below.

The field detection module 115 is configured to determine which fields are present in the target EMR 106. The field detection module 115 then reports a list of detected fields to the feature extraction module 114. The list of fields guides the feature extraction module 114 to extract the corresponding data from the endoscopic video data 102 and the metadata 104 for populating the listed fields.

Each field in the list of fields can be associated with a particular set of data requirements, such as data types, data formats, length limits or minimums, maximum and minimum allowed values, and so forth. The processing device 110 can further process the extracted data to conform to the specified format or data type of a given field. Generally, the target EMR 106 is associated with an interface 132 (e.g., an application programming interface or API) of an EMR system 130. The interface 132 provides requirements for the target EMR 106 to the processing device 110.

The computer-readable hardware storage device 111 (or computer-readable memory) can include any data storage technology type which is suitable to the local technical environment, including but not limited to semiconductor based memory devices, magnetic memory devices and systems, optical memory devices and systems, fixed memory, removable memory, disc memory, flash memory, dynamic random-access memory (DRAM), static random-access memory (SRAM), electronically erasable programmable read-only memory (EEPROM) and the like. In some implementations, the memory 111 (e.g., computer-readable hardware storage device) includes code-segment (or other executable logic) having executable instructions.

The processing device 110 can be communicatively coupled to the endoscopic processing unit 120 and configured to receive spatially arranged image data (e.g., video data) corresponding with one or more images captured by the imaging device. In some implementations, the processing device 110 includes a general purpose processor. In some implementations, the processing device 110 includes at least one applicable inference processor, accelerated processor which can be utilized in half, single, or double precision (16, 32, or 64 bit floating-point) calculation. The computer processor 110 can also include lots of compute unified device architecture (CUDA) cores, etc., or a combination of thereof. In some implementations, the computer processors 110 include a central processing unit (CPU). In some implementations, the processing device 110 includes at least one application specific integrated circuit (ASIC). The processing device 110 can also include general purpose programmable microprocessors, special-purpose programmable microprocessors, digital signal processors (DSPs), programmable logic arrays (PLAs), field programmable gate arrays (FPGA), special purpose electronic circuits, etc., or a combination thereof. The processing device 110 is configured to execute program code means such as the computer-executable instructions 112.

The data processing system can include a display unit (not shown) that is communicatively coupled to the computer processors 110 and configured to show results of the scoring and prediction processes described herein. The display unit can include an electronic display device. In some implementations, the display unit can be configured to act as a touchscreen display device. The display unit is configured to present a user interface. In some implementations, the user interface is a graphical user interface (GUI). The user interface is configured to allow a user of the data processing system 100 to interact with the data processing system 100 through graphical icons and visual indicators. The user interface can use a windows, icons, menus, pointer paradigm (WIMP) to allow a user to interact with the data processing system 100. In some implementations, the user interface cooperates with the endoscopic processing unit 120 to provide a user with a touchscreen GUI. Additionally, or alternatively, the user interface can include one or more input devices such as a mouse and/or keyboard communicatively coupled with the system 100. The user interface can also use a post-WIMP paradigm typically found in touchscreen-based GUIs. In some implementations, the user interface is configured to display images in the form of still photographs and/or videos. In some implementations, there is no display of the video data, and the structured data records are generated in a background process.

In an embodiment, the processing device 110 is configured to capture features of the endoscopy data 102 and metadata 104 in a stream and provide access to the EMR system 130, without determining any requirements of the EMR system 130 (e.g., without determining a list of fields). For example, the processing device 110 extracts features from the video data 102 and the metadata 104 and stores them in a feature storage 140 which can be accessed by the EMR system 130.

As previously described, types of information can include entry and exit times for the procedure, how many polyps were found and one or more characteristics of them. In some implementations, a human in the loop is still included to validate the generated record. Types of data can include when polyps appear and when they disappear in the video. The data processing system 100 is configured to capture images of polyps and potentially upload them to the EMR 106.

In some implementations, the data processing system 100 is configured to detect an instrument used during the procedure, such as forceps or a snare. For example, the data processing system 100 is configured to remove a bounding box generated around a polyp when a surgeon is acting on it. The data processing system 100 is configured to detect when in a surgery phase and detect a withdrawal phase of the procedure. The data processing system 100 is trained (e.g., using a machine learning system) to classify polyps and tools in the images.

To integrate into EMR records, a toolkit is provided to the EMR system 130. Using an API, a function library is provided for accessing particular features extracted from the video data. For example, rather than detect fields of the target EMR 104, the processing device 110 is configured to provide an output including unstructured text, possibly annotated with keywords. The data processing system 100 captures features and event data during the procedure, and the EMR system 130 can be configured to access the features as needed by requesting them through an interface 134 (e.g., an API). The feature data can be stored in the feature storage 140. In some implementations, the feature storage 140 includes a buffer.

FIG. 2A shows an example process for generating EMR data from endoscopy data. Generally, the data processing system (e.g., the data processing system 100 of FIG. 1) is configured to intake data for colonoscopy procedure or similar endoscopic procedure. The data processing system 100 is configured to auto populate fields of the EMR based on images 102 and metadata 104, as previously described, rather than require a physician to access the EMR directly and describe how the procedure went. As shown in FIG. 2A, the images and metadata 202 of the endoscopy are transformed into auto-generated EMR data 208.

The images and metadata 202 are received from the endoscopic processing unit 120. The feature extraction module 114 extracts feature data 204 from the video and image data 102 and the metadata 104. Here, the features are represented as blocks “A,” “B,” and “C.” The feature data 204 are generated based on events of the endoscopy, and these features can be combined to generate further data. For example, a start event corresponds to a start time feature, and a stop event corresponds to a stop time feature of the feature data 204. A duration of the procedure can be calculated by combining these two individual features.

As features are detected in the video data (e.g., a polyp is detected), the associated metadata (e.g., time elapsed for that image frame) can be associated with a newly extracted feature representing the polyp. For example, as a series of polyps are detected in the video data 102, each polyp is extracted as a feature (also called an event) of the feature data 204. In FIG. 2A, features A, B, and C can each represent a polyp detected in the image data 102. Each feature A, B, and C is associated with other data that are determined from analysis of the video data. For example, for the third polyp (e.g., feature or event “C”), a size value is determined by processing of the images including the third polyp detected. For example, for each video frame including the third polyp “C,” a size of the polyp is estimated (e.g., by border detection that is converted to a size based on a known distance from the polyp to the camera or a similar calculation). A series of frames shows the polyp with an average size when viewed from various angles. The processing device 110 stores the size average as feature data which can be used to populate the EMR 208 if requested by the EMR system 130.

Continuing with this example, transformed feature data 206 are generated from the processed image and video data 102 and metadata 104. For feature C, representing a third polyp, a time of detection is stored “Polyp 3 detected at 15 minutes into endoscopy.” For feature C, a location is stored “Polyp 3 is at lower GI tract.” For feature C, a size of the polyp is stored “Polyp 3 is 2 millimeters (mm) by 11 mm.” For feature C, additional data are stored “Polyp 3 disappears from view after a tool enters the frame and interacts with the polyp.” These transformed features 206a-d can be stored as plain text (as shown in FIG. 2A), or stored as numerical data or in any other format as desired.

In this example, the transformed feature 206d is not directly extracted from the video data 102. Rather, the processing device 110 is configured to analyze the video using one or more machine learning models. The feature extraction engine 114 can determine, based on the video, that a particular polyp (e.g., polyp 3) was present in the GI tract initially and then removed after a tool (e.g., a snare or forceps) was detected in one or more frames. The processing device 110 is configured to deduce that the tool removed the third polyp. The processing device 110 generates a transformed feature 206d indicating that this was detected, which may be useful for including in the EMR 208. In some implementations, a probability or confidence value (e.g., associated with a machine learning model output) can be included in the transformed feature and ultimately presented in the EMR. An example value 210 is shown in FIG. 2A as associated with transformed feature 206d and is included in auto-generated EMR 208.

The processing device 110 is configured to identify any complications arising from a procedure. For example, as in transformed feature 206d, the processing device can detect that Polyp 3 was unable to be removed even after tool has interacted with polyp. this indication is included in the Auto-Generated EMR 208 that Polyp 3 could not be removed. Alternatively, if the removal of Polyp 3 has led to other complications such as the perforation of the colon, could such data be recorded down in transformed feature 206c, and included in Auto-Generated EMR 208.

Auto-generated EMR 208 includes fields 1-4 each including respective values 208a-d that are generated from the respective transformed features 206a-d. Generally, the data included in the EMR 208 are determined based on a query configured by the EMR system 130, as subsequently described. In some implementations, the data processing system 100 is configured to determine what fields are included in EMR 208 and generate corresponding data to satisfy those fields with generated values.

The values 208a-d of the respective fields 1-4 of the EMR 208 can be based on the transformed features but need not be identical to the transformed features. In some implementations, features A, B, and C can be directly imported into the EMR 208. The report generated by the data processing system 100 can be formatted based on the query being generated by the EMR system 130. For example, when a size of the polyp is requested, the data processing system 100 can provide a numerical type output, a plain text stream output, and so forth based on the query.

The EMR 208 includes a structure that satisfies data quality rules and that enables computing systems to automatically retrieve details about the patient from the record based on the structure. For example, the data processing system can ensure that each of the fields of the record are populated with data in an expected format to prevent errors for processing the records for one or more applications. For example, the data processing system can ensure that each data record is associated with a valid key value 212 corresponding to a particular patient or consistent with a schema specified for storing the structured data record. For example, each video for a particular patient can be associated with a consistent key value 212 so that all of the data records for that patient can be retrieved from the system. The system can ensure that the key value does not conflict with one or more other key values associated with the patient which may occur when patient records are ingested from one or more target EMR/HER data sources. Additionally, each of the specific features of the structured data record, such as the features extracted from video data, can be associated with the key value in a consistent way so that each of these data items are accessible for analysis or automated analysis of the patient data. In this example, when processing the ER 208, the data processing system can extract all relevant data from the patient based on the key value 212 associated with that patient and can compare records over time based on this key value. The key value 212 can be used to associate different data records with one another and can be used to generate a structured data record in which all of the patients daily records are linked together, such as chronologically or using another structure.

FIG. 2B shows an example structured data record 220 including instances 208a-n of the autogenerated EMR 208. The structure of the data record enables analysis of patient data across many data records rather than relying on individual data records insolation for determining a patient's health status. In an example, particular feature associated with a particular patient record can be monitored over many different instances of patient records for that particular patient based on the key value or based on another identifier. For example, a patient may receive several endoscopies over the course of a year (or other time period). The structure data records described herein enable a particular feature extracted from that video data to be monitored and compared with prior instances for subsequent instances of video data required for that patient. For example, an anomaly or malignancy at a particular location the patient's colon can be flagged in a first video for generation of record 208a. The anomaly or malignancy is automatically associated with feature data such as the size/shape of the malignancy (e.g., Field 3), the location of the malignancy and the patient, the time associated with the data capture, and so forth. Data representing the same malignancy can be extracted from a subsequent video automatically for generation of record 208n. Extracted features of the malignancy (or lack thereof) from the subsequent video can be compared to the extracted features of the malignancy from the prior video to generate new feature data 222 representing comparative features of the malignancy, such as a growth/shrink rate, color change, and so forth. Based on the structure of the data records, the data processing system can automatically extract features from the video data or other data associated with patient and compare these extracted features to features of previous or subsequent instances of patient data at the feature level for generation of the comparative features.

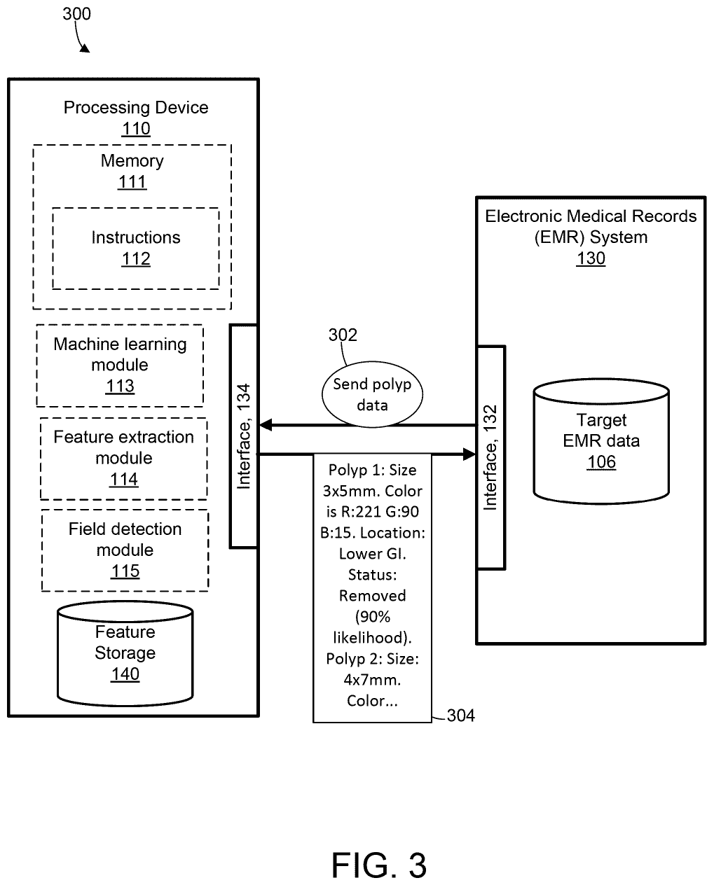

FIG. 3 shows an example environment 300 of a data processing system (e.g., data processing system 100 of FIG. 1) illustrating a query and response between the processing device 110 and the EMR system 130. In this example, the EMR system 130 either requests polyp data in a query 302, or the data processing system 100 pushes polyp data to the EMR system 130. Response 304 includes an unstructured list of polyps detected in the feature data or transformed feature data. The response 304 includes data associated with each polyp. In this example, each detected polyp is associated with a size value, a color value (e.g., an average color of the polyp), a location value, and a status value and respective probability based on a machine learning classifier output. While these four values are shown as an example output, any data previously described can be provided in response to a given query by the EMR system 130.

The interface 134 of the processing device 110 is associated with an API specifying a function library for structuring queries by the EMR system 130. The function library can specify a set of functions for querying the structured data record including querying each of the one or more fields of the data record or corresponding features represented in the one or more fields. The structured queries can be built from a library of relevant terms corresponding to the functions. For example, the set of functions can specify a query for extracting comparative features data for a particular patient or a particular type of malignancy or other particular feature of the structured data record. In another example, the queries can be structured based on a language model corresponding to dictionary that is built based on the features included in the structured data records. For example, the user can present a natural language query requesting a status on the progression of malignancy in a GI tract of the patient. The data processing system can associate the natural language query, based on a model, large language model, with similar language model, with one or more fields of the structured data record or one or more features included in the structured data record. Responsive to the query, the data processing system can automatically retrieve a set of data records and perform a comparison, if needed, to extract comparative feature data.

Extracting the one or more features from the image data can include automatically performing one or more image processing models or applying a machine learning model to the image data. In some implementations, the machine learning model is trained with images of GI tracts including images of malignancies or other anomalies. A set of machine learning models can each be trained to identify or extract a respective feature for populating a particular field of the EMR data (e.g., EMR 208). Responsive to a query, the data processing system can check the structured data records to determine whether the requested feature (e.g., a comparative feature) is present. If the feature is not already generated, the data processing system can automatically compare generated data records from different time instances and generate the comparative feature for presentation to the user.

In some implementations, the machine learning models can extract physical parameters or features from the image data (e.g., video data). For example, the physical features can include a color of the malignancy, an outer shape of the malignancy, an area of the malignancy, a location of the malignancy, or an orientation of the malignancy within the GI tract. The shape of the malignancy can refer to one or more shape signatures (such as curvature, overall smoothness, etc.) related to identifying a potential malignancy or type of anomaly in the GI tract.

In some implementations, the processing device 110 is configured to detect fields of the EMR 106 and provide data to satisfy those fields. In this embodiment, the interface 132 of the EMR system 130 is associated with an API specifying a function library for structuring output data for sending to the EMR system 130. The processing device 110 configures the output data based on the format, values, fields, and data types specified for a particular target EMR 104.

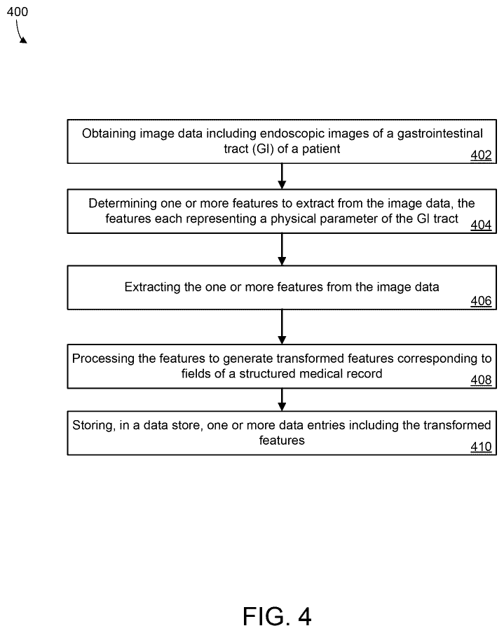

FIG. 4 shows a flow diagram showing a process 400 for generating EMR data from endoscopy data. The process 400 includes obtaining (402) image data including endoscopic images of a gastrointestinal tract (GI) of a patient. The process 400 includes determining (404) one or more features to extract from the image data, the features each representing a physical parameter of the GI tract. The process includes extracting (406) the one or more features from the image data. The process 400 includes processing (408) the features to generate transformed features corresponding to fields of a structured medical record. The process 400 includes storing (410), in a data store, one or more data entries including the transformed features.

In some implementations, extracting the one or more features from the image data comprises: applying a machine learning model to the image data, the machine learning model being trained with images of GI tracts; identifying, from the applying, a malignancy in the GI tract; determining one or more physical features of the malignancy; and outputting the one or more physical features as feature data.

In some implementations, the one or more physical features comprise a color of the malignancy, an outer shape of the malignancy, an area of the malignancy, a location of the malignancy, or an orientation of the malignancy.

In some implementations, the process 400 includes comparing the one or more physical features to corresponding one or more features extracted from other image data; and generating comparative features data.

In some implementations, the structured medical record comprises a set of instances of records each associated with a common identifier. The process 400 can include comparing data of a first field of a first instance of the instances of the records with a second field of a second instance of the instances of the records for generating comparative feature data.

In some implementations, the structured queries comprise a natural language query. The process 400 includes processing the natural language query using a language model trained based on a library of terms associated with the fields of the structured medical record.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 5 s. While the requested data need not be displayed (or initiated for display) instantaneously, it is displayed (or initiated for display) without any intentional delay, based on processing limitations of a described computing system and time required to, for example, gather, accurately measure, analyze, process, store, or transmit the data.

Events can include readings or measurements captured by a camera or other sensor. The readings or measurements can be analyzed by the data processing system, such as by using applications that can include modeling applications and machine learning. The analysis can be used to generate changes to settings of the cameras or other endoscopy equipment. In some implementations, values of parameters or other variables that are determined can be used automatically (such as through using rules) to implement changes in record generation. For example, outputs of the present disclosure can be used as inputs to other equipment and/or systems.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random-access memory (RAM), read only memory (ROM), phase change memory (PRAM), static random-access memory (SRAM), dynamic random-access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/-R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Any claimed implementation is applicable to at least a computer-implemented method; a non-transitory, computer-readable medium storing computer-readable instructions to perform the computer-implemented method; and a computer system comprising a computer memory interoperable coupled with a hardware processor configured to perform the computer-implemented method or the instructions stored on the non-transitory, computer-readable medium.

In the foregoing description, embodiments of the invention have been described with reference to numerous specific details that may vary from implementation to implementation. The description and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising,” in the foregoing description or following claims, what follows this phrase can be an additional step or entity, or a sub-step/sub-entity of a previously-recited step or entity.

What is claimed is:

1. A method for automatically generating a structured medical record from endoscopy data, the method comprising:
obtaining image data including endoscopic images of a gastrointestinal (GI) tract of a patient;
determining one or more features to extract from the image data, the features each representing a physical parameter of the GI tract;
extracting the one or more features from the image data;
processing the features to generate transformed features corresponding to fields of a structured medical record; and
storing, in a data store, one or more data entries including the transformed features, wherein the data store is configured to receive structured queries for the data entries in the data store and provide the data entries including the transformed features in response to receiving the structured queries.

2. The method of claim 1, wherein extracting the one or more features from the image data comprises:
applying a machine learning model to the image data, the machine learning model being trained with images of GI tracts;
identifying, from the applying, a malignancy in the GI tract;
determining one or more physical features of the malignancy; and
outputting the one or more physical features as feature data.

3. The method of claim 2, wherein the one or more physical features comprise a color of the malignancy, an outer shape of the malignancy, an area of the malignancy, a location of the malignancy, or an orientation of the malignancy.

4. The method of claim 2, further comprising:
comparing the one or more physical features to corresponding one or more features extracted from other image data; and
generating comparative features data.

5. The method of claim 1, wherein the structured medical record comprises a set of instances of records each associated with a common identifier, and wherein the method comprises:
comparing data of a first field of a first instance of the instances of the records with a second field of a second instance of the instances of the records for generating comparative feature data.

6. The method of claim 1, wherein the structured queries comprise a natural language query, and wherein the method comprises processing the natural language query using a language model trained based on a library of terms associated with the fields of the structured medical record.

7. A data processing system configured for automatically generating a structured medical record from endoscopy data, the data processing system comprising:
at least one processor; and
a memory storing instructions that, when executed by the at least one processor, cause the at least one processor to perform operations comprising:
obtaining image data including endoscopic images of a gastrointestinal tract (GI) of a patient;
determining one or more features to extract from the image data, the features each representing a physical parameter of the GI tract;
extracting the one or more features from the image data;
processing the features to generate transformed features corresponding to fields of a structured medical record; and
storing, in a data store, one or more data entries including the transformed features, wherein the data store is configured to receive structured queries for the data entries in the data store and provide the data entries including the transformed features in response to receiving the structured queries.

8. The data processing system of claim 7, wherein extracting the one or more features from the image data comprises:
applying a machine learning model to the image data, the machine learning model being trained with images of GI tracts;
identifying, from the applying, a malignancy in the GI tract;
determining one or more physical features of the malignancy; and
outputting the one or more physical features as feature data.

9. The data processing system of claim 8, wherein the one or more physical features comprise a color of the malignancy, an outer shape of the malignancy, an area of the malignancy, a location of the malignancy, or an orientation of the malignancy.

10. The data processing system of claim 8, the operations further comprising:
comparing the one or more physical features to corresponding one or more features extracted from other image data; and
generating comparative features data.

11. The data processing system of claim 7, wherein the structured medical record comprises a set of instances of records each associated with a common identifier, and wherein the operations further comprising:
comparing data of a first field of a first instance of the instances of the records with a second field of a second instance of the instances of the records for generating comparative feature data.

12. The data processing system of claim 7, wherein the structured queries comprise a natural language query, and wherein the operations comprise processing the natural language query using a language model trained based on a library of terms associated with the fields of the structured medical record.

13. One or more non-transitory computer readable media storing instructions for automatically generating a structured medical record from endoscopy data, the instructions, when executed by at least one processor, configured to cause the at least one processor to perform operations comprising:
obtaining image data including endoscopic images of a gastrointestinal tract (GI) of a patient;
determining one or more features to extract from the image data, the features each representing a physical parameter of the GI tract;
extracting the one or more features from the image data;
processing the features to generate transformed features corresponding to fields of a structured medical record; and
storing, in a data store, one or more data entries including the transformed features, wherein the data store is configured to receive structured queries for the data entries in the data store and provide the data entries including the transformed features in response to receiving the structured queries.

14. The data processing system of claim 13, wherein extracting the one or more features from the image data comprises:
applying a machine learning model to the image data, the machine learning model being trained with images of GI tracts;
identifying, from the applying, a malignancy in the GI tract;
determining one or more physical features of the malignancy; and
outputting the one or more physical features as feature data.

15. The data processing system of claim 14, wherein the one or more physical features comprise a color of the malignancy, an outer shape of the malignancy, an area of the malignancy, a location of the malignancy, or an orientation of the malignancy.

16. The data processing system of claim 14, the operations further comprising:
comparing the one or more physical features to corresponding one or more features extracted from other image data; and
generating comparative features data.

17. The data processing system of claim 13, wherein the structured medical record comprises a set of instances of records each associated with a common identifier, and wherein the operations further comprising:
comparing data of a first field of a first instance of the instances of the records with a second field of a second instance of the instances of the records for generating comparative feature data.

18. The data processing system of claim 13, wherein the structured queries comprise a natural language query, and wherein the operations comprise processing the natural language query using a language model trained based on a library of terms associated with the fields of the structured medical record.
